(19) United States
(12) Patent Application Publication
(10) Pub. No.: US 2016/0319222 A1
(43) Pub. Date: Nov. 3, 2016

(54) PEARLESCENT CLEANSING COMPOSITIONS

(71) Applicant: COLGATE-PALMOLIVE COMPANY, New York, NY (US)

(72) Inventor: Heriberto PEREZ-GARCIA, Santa Cruz, Meyehualco Iztapalapa (MX)

(73) Assignee: COLGATE-PALMOLIVE COMPANY, New York, NY (US)

(21) Appl. No.: 15/107,604

(22) PCT Filed: Dec. 26, 2013
(86) PCT No.: PCT/US2013/077781

§ 371 Date: Jun. 23, 2016

(51) Int. Cl. C11D 3/00; C11D 1/75; C11D 1/83; C11D 3/04; A61K 8/41; A61Q 19/10; A61K 8/20; A61Q 5/12; A61K 8/46; C11D 1/14; A61Q 5/02

(52) U.S. Cl. CPC C11D 3/0089; C11D 1/146; C11D 1/75; C11D 1/83; C11D 3/046; A61Q 5/02; A61Q 19/10; A61K 8/20; A61Q 5/12; A61K 8/463; A61K 8/416; A61K 2800/436; A61K 2800/596

(57) ABSTRACT

A composition comprising a surfactant and an amount of potassium chloride to impart pearlescence.

### BACKGROUND OF THE INVENTION

Liquid household and personal cleansing compositions such as laundry detergents, dish detergents, liquid hand soaps, body washes, shampoos, bath washes, and the like ideally possess a combination of excellent performance properties and attractive product appearance. Materials imparting pearlescence are sometimes added to these liquid cleansing compositions to achieve an aesthetically pleasing external appearance.

Prior art composition obtain pearlescence by the addition of additives such as fatty acids; fatty acid esters of polyethylene glycols; certain inorganic compounds such as mica, titanium oxide coated mica, coated silica; or polymer-based opacifiers based on polystyrene-acrylic copolymers. However, such additives can be costly and can have a negative impact on cleaning performance.

Therefore, those in the art of liquid cleansing compositions have been seeking new, cost effective formulations to help achieve excellent performance properties as well as an aesthetically pleasing pearlescence appearance.

### BRIEF SUMMARY

A cleansing composition comprising a surfactant and an amount of potassium chloride effective to impart pearlescence to the composition.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The cleansing composition comprises an amount of potassium chloride effective to impart pearlescence to the composition. In some embodiments the amount of potassium chloride 0.5 to 4% or 1 to 3%, or 2% by weight of the cleansing composition.

The cleansing composition includes one or more anionic surfactants, amphoteric surfactants, zwitterionic surfactants, nonionic surfactants, cationic surfactants, and combinations thereof to form a cleansing composition.

Surfactants can be included in any desired amount. Generally, at least 0.1 wt. % of the composition should be surfactant (a). Preferred minimums of at least 1, 3, 5, 7, 10, 20 and 30 wt. % surfactant(s) can be present in the composition. Maximum quantities of surfactant(s) depend upon the physical mixture of the composition being employed as well as the amount of additional components. Generally, no more than 95-97 wt. % surfactant(s) are present, more specifically no more than 90 weight % surfactant(s). Maximum quantities of 20, 30, 40, 50, 60, 70, 80, or 85 wt. % surfactant(s) can also be readily employed. In certain embodiments, the surfactant is present in an amount of 0.1 to 45, or 0.1 to 40, or 1 to 40, or 5 to 40, or 1 to 15, or 1 to 10, or 15 to 45, or 15 to 35, or 15 to 30, or 20 to 30, or 20 to 25, or 21 to 24, or 22 to 23 weight % of the composition depending on the type of composition.

Suitable anionic surfactants include, but are not limited to, those surface-active or detergent compounds that contain an organic hydrophobic group containing generally 6 to 26 or 8 to 26, or 6 to 22 or 10 to 18 carbon atoms in their molecular structure and at least one water-solubilizing group selected from sulfonate, sulfate, and carboxylate so as to form a water-soluble detergent. Usually, the hydrophobic group will comprise a C₈-C₂₂ alkyl, or acyl group. In some embodiments the anionic surfactant can be long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates and the like. These anionic surfactants can be alkoxylated, for example, ethoxylated, although alkoxylation is not required. Such surfactants can be employed in the form of water-soluble salts, can provide high foaming cleansing power, and the salt-forming cation usually is selected from sodium, potassium, ammonium, magnesium and mono-, di- or tri-C₂-C₃ alkanolammonium, with the sodium, magnesium and ammonium cations again being the usual ones chosen.

Suitable anionic surfactants include, but are not limited to, the sodium, potassium, ammonium, and ethanolammonium salts of linearC₈-C₁₆ alkyl benzene sulfonates, alkyl ether carboxylates, C₁₀-C₂₀ paraffin sulfonates, C₈-C₂₅ alpha olefin sulfonates, C₈-C₁₈ alkyl sulfates, C₈-C₁₈ alkyl ether sulfates and mixtures thereof.

Other suitable anionic surfactants include paraffin sulfonates, which may be monosulfonates or di-sulfonates and usually are mixtures thereof, obtained by sulfonating paraffins of 10 to 20 carbon atoms. Commonly used paraffin sulfonates are those of C₁₂-C₁₈ carbon atoms chains, and more commonly they are of C₁₄-C₁₇ chains. Paraffin sulfonates that have the sulfonate group(s) distributed along the paraffin chain are described in U.S. Pat. Nos. 2,503,280; 2,507,088; 3,260,744; and 3,372,188; and also in German Patent 735,096. Such compounds may be made to specifications and desirably the content of paraffin sulfonates outside the C₁₄-C₁₇ range will be minor and will be minimized, as will be any contents of di- or poly-sulfonates. Examples of paraffin sulfonates include, but are not limited to HOSTAPUR™ SAS30, SAS 60, SAS 93 secondary alkane sulfonates from Clariant, and BIO-TERGE™ surfactants from Stepan, and CAS No. 68037-49-0.

Examples of suitable other sulfonated anionic surfactants include higher alkyl mononuclear aromatic sulfonates, such as the higher alkylbenzene sulfonates containing 9 to 18 or 9 to 16 carbon atoms in the higher alkyl group in a straight or branched chain, or C₅-C₁₅ alkyl toluene sulfonates. In one embodiment, the alkylbenzene sulfonate is a linear alkylbenzene sulfonate having a higher content of 3-phenyl (or higher) isomers and a correspondingly lower content (well below 50%) of 2-phenyl (or lower) isomers, such as those sulfonates wherein the benzene ring is attached mostly at the 3 or higher (for example 4, 5, 6 or 7) position of the alkyl group and the content of the isomers in which the benzene ring is attached in the 2 or 1 position is correspondingly low. Materials that can be used are found in U.S. Pat. No. 3,320,174, especially those in which the alkyls are of 10 to 13 carbon atoms.

Other suitable anionic surfactants include the olefin sulfonates, including long-chain alkene sulfonates, long-chain hydroxyalkane sulfonates or mixtures of alkene sulfonates and hydroxyalkane sulfonates. These olefin sulfonate detergents may be prepared in a known manner by the reaction of sulfur trioxide (SO₃) with long-chain olefins containing 8 to 25, or 12 to 21 carbon atoms and having the formula RCH═CHR₁ where R is a higher alkyl group of 6 to 23 carbons and R₁ is an alkyl group of 1 to 17 carbons or hydrogen to form a mixture of sultones and alkene sulfonic acids which is then treated to convert the sultones to sulfonates. In one embodiment, olefin sulfonates contain from 14 to 16 carbon atoms in the R alkyl group and are obtained by sulfonating an a-olefin.

Examples of satisfactory anionic sulfate surfactants include alkyl sulfate salts and ether sulfate salts. Suitable anionic ether sulfate has the formula R(OC₂H₄)ₙ OSO₃M wherein n is 1 to 12, or 1 to 5, and R is an alkyl, alkylaryl, acyl, or alkenyl group having 8 to 18 carbon atoms, for example, an alkyl group of C₁₂-C₁₄ or C₁₂-C₁₆, and M is a solubilizing cation selected from sodium, potassium, ammonium, magnesium and mono-, di- and triethanol ammonium ions. Exemplary alkyl ether sulfates contain 12 to 15 carbon atoms in the alkyl groups thereof, e.g., sodium myristyl (3 EO) sulfate. Suitable alkylaryl ether sulfates include C₈-C₁₈ alkylphenyl ether sulfates containing from 2 to 6 moles of ethylene oxide in the molecule.

Other suitable anionic detergents include the C₉-C₁₅ alkyl ether polyethenoxyl carboxylates. Suitable alkyl ether polyethoxyl carboxylates may be prepared by condensing ethylene oxide with appropriate alkanol and reacting this reaction product with chloracetic acid to make the ether carboxylic acids as shown in U.S. Pat. No. 3,741,911 or with succinic anhydride or phtalic anhydride.

In some embodiments, anionic surfactants are present in the composition in an amount of 0 to 60%, or 0 to about 15, or 0.1 to 60, or 0.1 to 40, or 3 to 30, or 0.1 to 25, or 5 to 25, or 5 to 15, or 0.1 to 15 or 0.1 to 10, or 5 to 10% by weight of the cleansing composition.

Suitable nonionic surfactants utilized in this invention include the primary aliphatic alcohol ethoxylates, secondary aliphatic alcohol ethoxylates, alkylphenol ethoxylates and ethylene-oxide-propylene oxide condensates on primary alkanols, such a PLURAFAC™ surfactants (BASF) and condensates of ethylene oxide with sorbitan fatty acid esters such as the TWEEN™ surfactants (ICI), e.g., polysorbate 20. The nonionic synthetic organic detergents generally are the condensation products of an organic aliphatic or alkyl aromatic hydrophobic compound and hydrophilic ethylene oxide groups. Practically any hydrophobic compound having a carboxy, hydroxy, amido, or amino group with a free hydrogen attached to the nitrogen can be condensed with ethylene oxide or with the polyhydration product thereof, polyethylene glycol, to form a water-soluble nonionic detergent. Further, the length of the polyethenoxy chain can be adjusted to achieve the desired balance between the hydrophobic and hydrophilic elements.

The nonionic surfactant class includes the condensation products of a higher alcohol (e.g., an alkanol containing 8 to 18 carbon atoms in a straight or branched chain configuration) condensed with 5 to 30 moles of ethylene oxide, for example, lauryl or myristyl alcohol condensed with 16 moles of ethylene oxide (EO), tridecanol condensed with 6 to moles of EO, myristyl alcohol condensed with 10 moles of EO per mole of myristyl alcohol, the condensation product of EO with a cut of coconut fatty alcohol containing a mixture of fatty alcohols with alkyl chains varying from 10 to 14 carbon atoms in length and wherein the condensate contains either 6 moles of EO per mole of total alcohol or 9 moles of EO per mole of alcohol and tallow alcohol ethoxylates containing 6 EO to 11 EO per mole of alcohol.

Desirably suitable nonionic surfactants include the NEODOL™ ethoxylates (Shell Co.), which are higher aliphatic, primary alcohol containing 9-15 carbon atoms, such as C₉-C₁₁ alkanol condensed with 2 to 10 moles of ethylene oxide (NEODOL™ 91-2.5 OR -5 OR -6 OR -8), C₁₂-C₁₃ alkanol condensed with 6 to 7 moles ethylene oxide (NEODOL™ 23-6.5), C₁₂-C₁₅ alkanol condensed with 12 moles ethylene oxide (NEODOL™ 25-12), C₁₄-C₁₅ alkanol condensed with 13 moles ethylene oxide (NEODOL™ 45-13), and the like.

Additional satisfactory water soluble alcohol ethylene oxide condensates are the condensation products of a secondary aliphatic alcohol containing 8 to 18 carbon atoms in a straight or branched chain configuration condensed with 5 to 30 moles of ethylene oxide. Examples of commercially available nonionic detergents of the foregoing type are C₁₁-C₁₅ secondary alkanol condensed with either 9 EO (TERGITOL™ 15-S-9) or 12 EO (TERGITOL™ 15-S-12) marketed by Union Carbide.

Other suitable nonionic surfactants include the polyethylene oxide condensates of one mole of alkyl phenol containing from 8 to 18 carbon atoms in a straight- or branched chain alkyl group with 5 to 30 moles of ethylene oxide. Specific examples of alkyl phenol ethoxylates include, but are not limited to, nonyl phenol condensed with 9.5 moles of EO per mole of nonyl phenol, dinonyl phenol condensed with 12 moles of EO per mole of phenol, dinonyl phenol condensed with 15 moles of EO per mole of phenol and di-isoctylphenol condensed with 15 moles of EO per mole of phenol. Commercially available nonionic surfactants of this type include IGEPAL™ CO-630 (nonyl phenol ethoxylate) marketed by GAF Corporation.

Also among the satisfactory nonionic surfactants are the water-soluble condensation products of a C₈-C₂₀ alkanol with a mixture of ethylene oxide and propylene oxide wherein the weight ratio of ethylene oxide to propylene oxide is from 2.5:1 to 4:1, or 2.8:1 to 3.3:1, with the total of the ethylene oxide and propylene oxide (including the terminal ethanol or propanol group) being from 60-85%, or 70-80%, by weight. Such detergents are commercially available from BASF and a particularly preferred surfactant is a C₁₀-C₁₆ alkanol condensate with ethylene oxide and propylene oxide, the weight ratio of ethylene oxide to propylene oxide being 3:1 and the total alkoxy content being 75% by weight.

Condensates of 2 to 30 moles of ethylene oxide with sorbitan mono- and tri-C₁₀-C₂₀ alkanoic acid esters having a HLB of 8 to 15 also may be employed as the nonionic detergent ingredient in the described composition. These surfactants are well known and are available from Imperial Chemical Industries under the TWEEN™ trade name. Suitable surfactants include, but are not limited to, polyoxyethylene (4) sorbitan monolaurate, polyoxyethylene (4) sorbitan monostearate, polyoxyethylene (20) sorbitan trioleate and polyoxyethylene (20) sorbitan tristearate.

Other suitable water-soluble nonionic surfactants are polypropylene glycol ethoxylates marketed under the trade name PLURONIC™. The compounds are formed by condensing ethylene oxide with a hydrophobic base formed by the condensation of propylene oxide with propylene glycol. The molecular weight of the hydrophobic portion of the molecule is of the order of 950 to 4000 or 200 to 2,500. The addition of polyoxyethylene radicals to the hydrophobic portion tends to increase the solubility of the molecule as a whole so as to make the surfactant water-soluble. The molecular weight of the block polymers varies from 1,000 to 15,000 and the polyethylene oxide content may comprise 20% to 80% by weight. In one embodiment, these surfactants will be in liquid form and satisfactory surfactants are available as grades L 62 and L 64.

In some embodiments, nonionic surfactants are present in the composition in an amount of 0 to 25%, or 0 to about 10, or 0 to 2, or 0.1 to 20, or 0.1 to 10, or 0.1 to 5 or 0.5 to 1.5% by weight of the cleansing composition.

Alkyl polysaccharides surfactants, which can be used in the instant composition, have a hydrophobic group containing from 8 to 20 carbon atoms, or from 10 to 16 carbon atoms, or from 12 to 14 carbon atoms, and polysaccharide hydrophilic group containing from 1.5 to 10, or from 1.5 to 4, or from 1.6 to 2.7 saccharide units (e.g., galactoside, glucoside, fructoside, glucosyl, fructosyl; and/or galactosyl units). Mixtures of saccharide moieties may be used in the alkyl polysaccharide surfactants. The number x indicates the number of saccharide units in a particular alkyl polysaccharide surfactant. For a particular alkyl polysaccharide molecule x can only assume integral values. In any physical sample of alkyl polysaccharide surfactants there will be in general molecules having different x values. The physical sample can be characterized by the average value of x and this average value can assume non-integral values. In this specification the values of x are to be understood to be average values. The hydrophobic group (R) can be attached at the 2-, 3-, or 4-positions rather than at the 1-position, (thus giving e.g. a glucosyl or galactosyl as opposed to a glucoside or galactoside). However, attachment through the 1-position, i.e., glucosides, galactoside, fructosides, etc., is preferred. In one embodiment, the additional saccharide units are predominately attached to the previous saccharide unit's 2-position. Attachment through the 3-, 4-, and 6-positions can also occur. Optionally and less desirably there can be a polyalkoxide chain joining the hydrophobic moiety (R) and the polysaccharide chain. The preferred alkoxide moiety is ethoxide.

Typical hydrophobic groups include alkyl groups, either saturated or unsaturated, branched or unbranched containing from 8 to 20, or from 10 to 18 carbon atoms. In one embodiment, the alkyl group is a straight chain saturated alkyl group. The alkyl group can contain up to 3 hydroxy groups and/or the polyalkoxide chain can contain up to 30, or less than 10, alkoxide moieties.

Suitable alkyl polysaccharides include, but are not limited to, decyl, dodecyl, tetradecyl, pentadecyl, hexadecyl, and octadecyl, di-, tri-, tetra-, penta-, and hexaglucosides, galactosides, lactosides, fructosides, fructosyls, lactosyls, glucosyls and/or galactosyls and mixtures thereof.

The alkyl monosaccharides are relatively less soluble in water than the higher alkyl polysaccharides. When used in a mixture with alkyl polysaccharides, the alkyl monosaccharides are solubilized to some extent. The use of alkyl monosaccharides in admixture with alkyl polysaccharides is a preferred mode of carrying out the invention. Suitable mixtures include coconut alkyl, di-, tri-, tetra-, and pentaglucosides and tallow alkyl tetra-, penta-, and hexaglucosides.

“Alkyl polysaccharide surfactant” is intended to represent both the glucose and galactose derived surfactants and the alkyl polysaccharide surfactants. Throughout this specification, “alkyl polyglucoside” is used to include alkyl polyglycosides because the stereochemistry of the saccharide moiety is changed during the preparation reaction.

Suitable alkyl polyglucosides include APG 625 glycoside manufactured by the Henkel Corporation of Ambler, Pa. APG 625 is a nonionic alkyl polyglycoside characterized by the formula:

CₙH₂ₙ₊₁O(C₆H₁₀O₅)ₓH

wherein n=10 (2%); n=122 (65%); n=14 (21-28%); n=16 (4-8%) and n=18 (0.5%) and x (degree of polymerization)=1.6. APG 625 has: a pH of 6 to 10 (10% of APG 625 in distilled water); a specific gravity at 25° C. of 1.1 g/ml; a density at 25° C. of 9.1 lbs/gallon; a calculated HLB of 12.1 and a Brookfield viscosity at 35° C., 21 spindle, 5-10 RPM of 3,000 to 7,000 cps.

In some embodiments, the alkyl polysaccharide surfactants are present in the composition in an amount of 0 to 25%, or 0 to about 10, or 0 to 2, or 0.1 to 20, or 0.1 to 10, or 0.1 to 5 or 0.1 to 2% by weight of the cleansing composition.

Amphoteric or zwitterionic surfactants may also be included in the composition. These surfactants are typically characterized by a combination of high surfactant activity, lather forming and mildness. Suitable zwitterionic or amphoteric surfactants include betaines and sultaines, such as, a water soluble betaine having the general formula

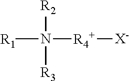

wherein X⁻ is selected from COO⁻ and SO₃⁻ and R₁ is an alkyl group having 10 to 20 carbon atoms, or 12 to 16 carbon atoms, or the amido radical:

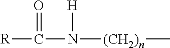

wherein R is an alkyl group having 9 to 19 carbon atoms and n is the integer 1 to 4; R₂ and R₃ are each alkyl groups having 1 to 3 carbons and in one embodiment, 1 carbon; R₄ is an alkylene or hydroxyalkylene group having from 1 to 4 carbon atoms and, optionally, one hydroxyl group. Typical alkyldimethyl betaines include, but are not limited to, decyl dimethyl betaine or 2-(N-decyl-N, N-dimethyl-ammonia) acetate, coco dimethyl betaine or 2-(N-coco N, N-dimethylammonia) acetate, myristyl dimethyl betaine, palmityl dimethyl betaine, lauryl dimethyl betaine, cetyl dimethyl betaine, stearyl dimethyl betaine, etc. The amidobetaines similarly include, but are not limited to, cocoamidoethylbetaine, cocoamidopropyl betaine and the like. The amidosulfobetaines include, but are not limited to, cocoamidoethylsulfobetaine, cocoamidopropyl sulfobetaine and the like. In one embodiment, the betaine is coco (C₈-C₁₈) amidopropyl dimethyl betaine. Three examples of betaine surfactants that can be used are EMPIGEN™ BS/CA from Albright and Wilson, REWOTERIC™ AMB 13 and Goldschmidt Betaine L7.

Other suitable zwitterionic surfactants include amine oxides. An amine oxide is depicted by the formula:

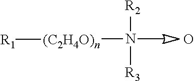

wherein R₁ is an alkyl, 2-hydroxyalkyl, 3-hydroxyalkyl, or 3-alkoxy-2-hydroxypropyl radical in which the alkyl and alkoxy, respectively, contain from 8 to 18 carbon atoms; R₂ and R₃ are each methyl, ethyl, propyl, isopropyl, 2-hydroxyethyl, 2-hydroxypropyl, or 3-hydroxypropyl; and n is from 0 to 10. In one embodiment, the amine oxides are of the formula:

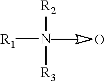

wherein R₁ is a C₁₂₋₁₈ alkyl and R₂ and R₃ are methyl or ethyl. The above ethylene oxide condensates, amides, and amine oxides are more fully described in U.S. Pat. No. 4,316,824. In another embodiment, the amine oxide is depicted by the formula:

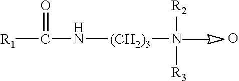

wherein R₁ is a saturated or unsaturated alkyl group having 6 to 24 carbon atoms, R₂ is a methyl group, and R₃ is a methyl or ethyl group. A preferred amine oxide is cocoamidopropyl-dimethylamine oxide.

In some embodiments, amphoteric or zwitterionic surfactants are present in the composition in an amount of 0 to 30%, or 0 to about 10, or 0 to 2, or 0.1 to 30, or 0.1 to 20, or 0.1 to 10 or 0.1 to 5, or 4 to 6% by weight of the cleansing composition.

Cationic surfactants can also be included in the composition. Examples of cationic surfactants include, but are not limited to any quaternium or polyquaternium compound. Cationic surfactants can be included at any desired level. It may be desirable to include the cationic surfactant(s) in an amount of up to 25% by weight, or up to 10% by weight, or up to 3% by weight, of the total composition. In some embodiments, cationic surfactants are present in the composition in an amount of 0 to 25%, or 0 to about 10, or 0 to 2, or 0.1 to 20, or 0.1 to 10, or 0.1 to 5 or 0.1 to 2, or 0.1 to 0.3% by weight of the cleansing composition.

One desirable surfactant for the cleaning composition is an alkyl benzene sulfonate surfactant. Another desirable surfactant is a surfactant system of a combination of an alkyl ether sulfonate, and a zwitterionic surfactant. Yet another desirable is a surfactant system of a combination of an alkyl benzene sulfonate, an alkyl ether sulfonate, and a zwitterionic surfactant In an embodiment it may be desirable to include the alkyl benzene sulfonate salt surfactant(s) in an amount of 1% to 40% by weight, or 3% by weight to 30% by weight, or 5% to 20% by weight of the total composition, with attention to the relative ratio various salts (of applicable), e.g., sodium to magnesium salts, as described above. In an embodiment it may be desirable to include the alkyl ether sulfate surfactant(s) in an amount of 5% to 40% by weight, or 10% to 25% by weight, or 7% to 19% by weight. In an embodiment it may be desirable to include the amine oxide surfactant(s) in an amount of up to 25% by weight, or 5% to 20% by weight, or 2% to 10% by weight.

A preferred surfactant comprises a mixture of an anionic and a nonionic surfactant. In one embodiment the anionic surfactant is an alkyl sulfate surfactant and the nonionic surfactant is an amine oxide surfactant. In one embodiment the alkyl sulfate surfactant is ethoxylated, i.e., is an ether sulfate. In a particular embodiment the ether sulfate surfactant is ammonium laureth sulfate and the amine oxide surfactant is lauryl myristyl diamine oxide. In some embodiments the amount of alkyl sulfate or ether sulfate surfactant, e.g., ammonium laureth sulfate, is 6 to 22% or 10 to 20% by weight of the composition. In some embodiments the amount of amine oxide surfactant, e.g., lauryl myristyl diamine oxide, is 2.5 to 6% or 3 to 4.5% by weight of the composition. In some embodiments the amount of binary surfactant consisting of a combination of an alkyl sulfate or ether sulfate surfactant, e.g., ammonium laureth sulfate, and an amine oxide surfactant, e.g., lauryl myristyl diamine oxide, comprises 9 to 25% or 22 to 23% by weight of the composition.

In addition to the surfactant and the potassium chloride, the composition on the invention may include other ingredients which are collectively referred to herein as a “carrier material”.

A suspending agent can be included to structure the surfactant to aid in suspending particles. Suspending agents are any material that increases the ability of the composition to suspend material. Examples of suspending agents include, but are not limited to, synthetic structuring agents, polymeric gums, polysaccharides, pectin, alginate, arabinogalactan, carrageen, gellan gum, xanthum gum, guar gum, rhamsan gum, furcellaran gum, and other natural gum. A synthetic structuring agent in one embodiment is a polyacrylate. One acrylate aqueous solution used to form a stable suspension of the solid particles is manufactured by Lubrizol as CARBOPOL™ resins, also known as CARBOMER™, which are hydrophilic high molecular weight, crosslinked acrylic acid polymers. In one embodiment, the polymer is CARBOPOL™ Aqua SF-1. Other polymers that can be used include, but are not limited to, CARBOPOL™ Aqua 30, CARBOPOL™ 940 with a molecular weight of approximately 4,000,000, and CARBOPOL™ 934 with a molecular weight of approximately 3,000,000.

The suspending agents can be used alone or in combination. The amount of suspending agent can be any amount that provides for a desired level of suspending ability. In one embodiment, the suspending agent is present in an amount of 0.01 to 15% by weight of the composition. In other embodiments, the amount of suspending agent is 1% to 10%.

In another embodiment, glycerin may be included in the composition. The glycerin can be included in any desired amount to provide a desired level of moisturization. In one embodiment, the glycerin is present in an amount of 0 to 8% by weight. In other embodiments, the glycerin can be present at 1, 1.5, 2, 2.5, 3, 3.5, 4, 4.5, 5, 5.5, 6, 6.5, 7, or 7.5% by weight.

Antioxidants such as, for example, butylated hydroxytoluene (BHT) and Vitamin A, C & E or their derivatives may be used advantageously in e.g., amounts of 0.01% or higher if appropriate In one embodiment, the composition can contain vitamin E and/or vitamin E acetate (a vitamin E precursor). In one embodiment, the amount of vitamin E and/or vitamin E acetate in the composition is 0.005 weight % to 0.5 weight %. In other embodiments, the amount is at least 0.01, at least 0.05, or at least 0.1 weight %. In other embodiments, the amount is less than 0.5 weight %. Examples of preservatives include dimethyloldimethylhydantoin (Glydant XL1000), parabens, sorbic acid and the like.

The cleansing composition can include thickeners, such as carboxymethylcellulose, magnesium aluminum silicate, hydroxyethylcellulose, methylcellulose, carbopols, glucamides, or Antil® from Rhone Poulenc. Other thickeners which may be used include Amerchol Polymer HM 1500 (Nonoxynyl Hydroethyl Cellulose); Glucam DOE 120 (PEG 120 Methyl Glucose Dioleate); Rewoderm® (PEG modified glyceryl cocoate, palmate or tallowate) from Rewo Chemicals; Antil® 141 (from Goldschmidt). In one embodiment the composition also utilizes, as a thickening agent, a blend of PEG-150 distearate and PPG-2 hydroxyethyl cocamide for countering a decrease in viscosity associated with the concentrations of moisturizing agents utilized in some embodiments of a moisturizing body wash composition. The PEG-150 distearate and the PPG-2 hydroxyethyl cocamide are available as a mixture from Uniqema under the trade name PROMIDIUM™ LTS. The amount of thickener can be 0 to 20, or 0.1 to 20, or 0.1 to 10, or 0.1 to 5% by weight of the cleansing composition.

The cleansing compositions described herein are preferably in the form of liquids in which water is the principal diluent. Alternatively, although less preferred, other solvents such as alcohols may be utilized. The level of water in the cleansing composition is 0 to 99% by weight, or from 30% to 99% by weight, or 50% to 90% by weight or from 70% to 90% by weight, or from 80% to 90% by weight.

The composition may include one or more of fragrance compounds. The composition may include 0.01% to 6% of a fragrance compound or 0.04 to 6%, based on the total solid weight of the composition.

The composition may include an antimicrobial. Examples of antimicrobials are 2-hydroxy-4,2′4′ trichlorodiphenylether (DP300), lactic acid, quaternary ammonium compounds such as Triclosan (2,4,4′-trichloro-2′-hydroxydiphenyl ether), DMDM hydantoin, formaldehyde and/or imidazolidinyl urea, and/or their equivalents, and the like, e.g., in amounts of 0 to 2, or 0.0001 to 2% by weight.

Other specific examples of optional ingredients include organic solvents, such as ethanol; sequestering agents, such as tetrasodium ethylenediaminetetraacetate (EDTA), EHDP or mixtures in an amount of 0.01 to 1%, preferably 0.01 to 0.05%; and coloring agents, additional opacifiers/pearlizers such as zinc stearate, magnesium stearate, TiO₂, EGMS (ethylene glycol monostearate) or Lytron 621 (Styrene/Acrylate copolymer); all of which are useful in enhancing the appearance or cosmetic properties of the product.

The compositions may also comprise coconut acyl mono- or diethanol amides as suds boosters, and strongly ionizing salts such as sodium chloride and sodium sulfate may also be used to advantage.

The invention therefore provides, in a first embodiment a cleansing composition comprising a surfactant and an amount of potassium chloride effective to impart pearlescence to the composition (Composition 1), e.g.,
1.1. Any of the foregoing compositions wherein the surfactant is anionic, nonionic, amphoteric/zwitterionic, cationic or a mixture thereof;
1.2. The foregoing composition, wherein surfactant comprises a mixture of an anionic and a nonionic surfactant;
1.3. Composition 1.1 or 1.2 wherein the anionic surfactant is present in an amount of 0.1 to 60, or 0.1 to 40, or 3 to 30, or 0.1 to 25, or 5 to 25, or 5 to 15, or 0.1 to 15 or 0.1 to 10, or 5 to 10% by weight of the cleansing composition;
1.4. Composition 1.1, 1.2 or 1.3 wherein the nonionic surfactant is present in an amount of 0.1 to 20, or 0.1 to 10, or 0.1 to 5 or 0.5 to 1.5% by weight of the cleansing composition;
1.5. Compositions 1.1-1.4 wherein the anionic surfactant is an alkyl sulfate or ether sulfate surfactant;
1.6. Compositions 1.1-1.5 wherein the anionic surfactant is ammonium laureth sulfate;
1.7. Composition 1.6 wherein the amount of ammonium laureth sulfate is 6 to 22% or 10 to 20% by weight of the cleansing composition;
1.8. Compositions 1.1-1.7 wherein the nonionic surfactant is an amine oxide surfactant;
1.9. Compositions 1.1-1.8 wherein the nonionic surfactant is lauryl/myristyl amine oxide;
1.10. Composition 1.9 wherein the amount of lauryl/myristyl amine oxide is 2.5 to 6% or 3 to 4.5% by weight of the cleansing composition;
1.11. Any of the foregoing compositions wherein the surfactant is a combination of ammonium laureth sulfate and lauryl/myristyl amine oxide;
1.12. The foregoing composition wherein the amount of the combination of ammonium laureth sulfate and lauryl/myristyl amine oxide is 9 to 25% or 22 to 23% by weight of the cleansing composition;
1.13. Any of the foregoing compositions wherein the amount of potassium chloride is 0.5 to 4% or 1 to 3%, or 2% by weight of the cleansing composition;
1.14. Any of the foregoing compositions comprising a carrier;
1.15. The foregoing composition wherein the carrier comprises a suspending agent;
1.16. Compositions 1.14-1.15 wherein the carrier comprises water, e.g., in an amount of 0 to 99% by weight, or from 30% to 99% by weight, or 50% to 90% by weight or from 70% to 90% by weight, or from 80% to 90% by weight of the cleansing composition;
1.17. Compositions 1.14-1.16 wherein the carrier comprises one or a mixture of glycerin, an antimicrobial agent, creatine, a moisturizer, keratin, a thickener, an antioxidant, a sequestering agent, a fragrance, a colorant, or an organic solvent;
1.18. Any of the foregoing compositions, wherein the composition is a dish detergent or a laundry detergent;
1.19. Any of the foregoing compositions wherein the composition is a body wash, a shower gel, a liquid hand soap, a shampoo, or hair conditioner.

The compositions and the methods of producing such compositions herein may be formulated and carried out such that they will have a pH of 4 to 8.5, and, in some embodiments, 5 to 7. Techniques for controlling pH at recommended usage levels include the use of buffers, alkali, acids, etc., and are well known to those skilled in the art. Optional pH adjusting agents can include, but are not limited to citric acid, succinic acid, phosphoric acid, sodium hydroxide, sodium carbonate, and the like.

The composition can be used to clean household surfaces, e.g. dishes, and fabrics. The compositions can also be used to clean and/or moisturize skin, hair, and/or nails. The composition can be applied to skin, hair, and/or nails. If the composition is a rinse off composition, the composition is rinsed off. The composition can be left on for any desired amount of time. The composition can be used by itself or can be included in any product that contacts fabrics, household surfaces, skin, including hair, and/or nails. The composition can be in the form of a body wash, a shower gel, a liquid hand wash, a shampoo, a conditioner, a dishwashing liquid, a laundry detergent, a skin lotion, a sunscreen, a bubble bath, and the like.

The composition can also be used to apply a substance to a substrate. The substance is included in the composition, and the composition is applied to a substrate. The substrate can be any desired substrate. In one embodiment, the substrate can be skin, hair, and/or nails. In another embodiment the surface is a fabric, dish, plastic, metal, porcelain, or glass. The substance can be any substance that is attracted to the composition. In one embodiment, the substance is chosen from fragrances, sunscreen, pigments, insect repellents, and/or hydrophobic materials.

In one embodiment the compositions of the invention are personal cleansing compositions. The personal cleansing compositions of the present invention preferably exhibit enhanced skin feel, low skin irritation, low skin drying, good cleansing ability, good foaming, and good rinsability characteristics/properties. Preferably, the present personal cleansing compositions also do not leave the skin feeling tacky or tight during drying. The cleansing compositions may be utilized alone, as skin or body washes, or may be utilized with soaps, thickeners, additives, or the like to produce various liquid soap-based or synthetic hand cleansers, face cleansers, body washes, bath soaps, bath beads, shower gels, shampoos, dish washing liquids, denture cleansers, and the like.

The invention includes a method for cleansing a substrate comprising contacting the substrate with the cleansing composition of the invention under conditions sufficient to cleanse said substrate. The substrate can be any desired substrate. In one embodiment, the substrate can be skin, hair, and/or nails. In another embodiment the substrate is a fabric, dish, plastic, metal, porcelain, or glass. The composition may be contacted with the substrate for various times, depending in the product form, e.g., 1 second up to 24 hours. The composition may be diluted with water during normal application. The composition may be applied with an applicator, e.g., cloth or sponge, or may be applied with pressure or agitation.

The invention also include use of potassium chloride to make a surfactant composition pearlescent.

The following examples are merely illustrative and do not in any way limit the scope of the invention as described and claimed. Unless otherwise listed, the amounts are % by weight of the composition based on the active weight of the material.

### Example 1

The following composition was prepared by mixing of the ingredients. The amounts are based on the as supplied amounts.

| Material | Weight % |
|---|---|
| Surfactant: | 22.25 |
| 18% ammonium laureth sulfate; | |
| 4.25% lauryl myristyl diamine oxide | |
| Potassium chloride | 2 |
| Water and minors | Q.S. |

The compositions can be made by mixing of the ingredients. The composition is pearlescent when viewed by a non-colorblind, human eye with 20/20 vision or corrected to 20/20 vision and viewed at a distance of 30 cm. Pearlescent is used in its ordinary dictionary meaning of having a mother of pearl luster (Oxford Dictionary).

1. A cleansing composition comprising a surfactant and an amount of potassium chloride effective to impart pearlescence to the composition.

2. The composition of claim 1, wherein surfactant comprises a mixture of an anionic and a nonionic surfactant.

3. The composition of claim 2, wherein the anionic surfactant is present in an amount of 0.1 to 60 weight %, optionally, 0.1 to 40, 3 to 30, 0.1 to 25, 5 to 25, 5 to 15, 0.1 to 15 0.1 to 10, or 5 to 10% by weight of the cleansing composition.

4. The composition of any of claim 2, wherein the nonionic surfactant is present in an amount of 0.1 to 20 weight %, optionally 0.1 to 10, 0.1 to 5, or 0.5 to 1.5% by weight of the cleansing composition.

5. The composition of claim 2, wherein the anionic surfactant is an alkyl sulfate or alkyl ether sulfate surfactant.

6. The composition of claim 2, wherein the anionic surfactant is ammonium laureth sulfate.

7. The composition of claim 2, wherein the nonionic surfactant is an amine oxide surfactant.

8. The composition of claim 2, wherein the nonionic surfactant is lauryl/myristyl amine oxide.

9. The composition of claim 1 wherein the surfactant is a combination of ammonium laureth sulfate and lauryl/myristyl amine oxide in an amount of 9 to 25 weight % optionally 22 to 23% by weight of the cleansing composition.

10. The composition of claim 1 wherein the amount of potassium chloride is 0.5 to 4%, optionally, 1 to 3% or 2% by weight of the cleansing composition.

11. The composition of claim 1 further comprising water in an amount of 30 to 99 weight %, optionally 50 to 90% or 70 to 90% by weight of the cleansing composition.

12. The composition of claim 1 in the form of a dish washing liquid, laundry detergent, body wash, shower gel, liquid hand soap, shampoo, or conditioner.

13. A method for cleansing a substrate comprising contacting the substrate with the cleansing composition of claim 1 under conditions sufficient to cleanse said substrate.

14. (canceled)
